(19) United States
(12) Patent Application Publication
(10) Pub. No.: US 2025/0276217 A1
(43) Pub. Date: Sep. 4, 2025

(54) PREDICTION DEVICE, PREDICTION METHOD, AND NON-TRANSITORY COMPUTER-READABLE STORAGE MEDIUM

(71) Applicant: HONDA MOTOR CO., LTD., Tokyo (JP)

(72) Inventors: Koji AKATSUKA, Saitama (JP); Yasushi IKEUCHI, Saitama (JP)

(21) Appl. No.: 18/986,186

(22) Filed: Dec. 18, 2024

(30) Foreign Application Priority Data
Mar. 1, 2024 (JP) 2024-031343

(51) Int. Cl. A63B 24/00; G01L 5/13

(52) U.S. Cl. CPC A63B 24/0084; A63B 24/0062; G01L 5/13

(57) ABSTRACT

A prediction device for predicting a run time of a target person who competes in a race by driving a wheelchair, the prediction device includes: a storage device configured to store a measured value of at least one item regarding a physical ability of an athlete who competes in the race and the run time of the athlete that were acquired in the past such that the measured value of the at least one item and the run time are associated with each other; and a processor configured to acquire an expected value corresponding to the at least one item, to calculate a predicted value of the run time corresponding to the expected value, and to output the predicted value.

### TECHNICAL FIELD

The present invention relates to a prediction device, a prediction method, and a prediction program (stored in a non-transitory computer-readable storage medium) for predicting a run time of an athlete who competes in a race by driving a wheelchair.

### BACKGROUND ART

Regarding a wheelchair for racing used in a track race, a marathon, and the like, there is a demand for accurately capturing the applied position of the driving force from the occupant to the hand rim for the training of the occupant. To meet such a demand, JP7157723B2 discloses a driving force applied position estimation system that can accurately estimate an applied position of the driving force from the occupant of the wheelchair.

In the track races, marathons, and the like in which wheelchairs for racing are used, the athletes compete for the fastest run time. The applied position of the driving force obtained by the driving force applied position estimation system of JP7157723B2 can be an item that determines the run time. However, the run time is considered to also depend on items that characterize the physical ability other than the driving force applied position.

The inventors of the present application considered that it would be possible to provide suggestions on the training to the athlete or the coach if how the training will change the run time can be predicted by inputting the post-training expected values of the items that characterize the physical ability of the athlete.

### SUMMARY OF THE INVENTION

In view of the foregoing background, a primary object of the present invention is to provide a prediction device, a prediction method, and a prediction program which can properly predict a run time of an athlete who competes in a race by driving a wheelchair based on an expected value of an item characterizing the physical ability of the athlete.

To achieve the above object, one aspect of the present invention provides a prediction device (21) for predicting a run time of a target person who competes in a race by driving a wheelchair (1), the prediction device comprising: a storage device (24) configured to store a measured value of at least one item regarding a physical ability of an athlete who competes in the race and the run time of the athlete that were acquired in the past such that the measured value of the at least one item and the run time are associated with each other; and a processor (22) configured to acquire an expected value corresponding to the at least one item, to calculate a predicted value of the run time corresponding to the expected value, and to output the predicted value.

According to this aspect, it is possible to refer to the measured value of the at least one item regarding the physical ability of the athlete and the run time of the athlete that were acquired in the past, and therefore, the run time of the athlete can be properly predicted based on the expected value of the item(s) characterizing the physical ability of the athlete.

Preferably, the at least one item includes at least one of six abilities consisting of a speed ability, a movable angle ability, an endurance ability, a rhythm ability, a recognition ability, and a power ability.

According to this aspect, it is possible to set an item characterizing the physical ability of the athlete.

Preferably, the movable angle ability is acquired from an angle difference from a contact start position where the athlete starts touching a hand rim of the wheelchair to a contact end position where contact with the hand rim ends.

According to this aspect, the movable angle ability can be properly evaluated.

Preferably, the endurance ability is acquired by making the athlete run around a track multiple times in the wheelchair and calculating a degree of increase in time it takes to run around the track.

According to this aspect, the endurance ability can be properly evaluated.

Preferably, the rhythm ability is acquired by calculating a correlation coefficient of waveforms each showing a time change of a driving force applied by the athlete when rotating a rear wheel of the wheelchair one turn or a component of the driving force in a tangential direction of a hand rim of the wheelchair.

According to this aspect, the rhythm ability can be properly evaluated.

Preferably, the recognition ability is acquired from a ratio between a tangential force which is a component of a driving force exerted by the athlete in a tangential direction of a hand rim of the wheelchair and a pressing force which is a resultant force of components of the driving force excluding the component in the tangential direction of the hand rim.

According to this aspect, the recognition ability can be properly evaluated.

Preferably, the at least one item includes at least four of the six abilities consisting of the speed ability, the movable angle ability, the endurance ability, the rhythm ability, the recognition ability, and the power ability.

According to this aspect, the items characterizing the physical ability of the athlete can be set more properly.

Preferably, the prediction device comprises the wheelchair (1A) provided with a vehicle body frame (3), a rear wheel (13), a hand rim (15) provided on the rear wheel, and a sensor (43, 44) configured to detect a force applied to the hand rim and a rotation position of the hand rim, and the prediction device acquires the measured value by making the athlete run using the wheelchair.

According to this aspect, the measured value can be properly acquired.

Preferably, the storage device is configured to store a run log in which the measured value of each of the at least one item and the run time corresponding thereto are recorded so as to be associated with each other for each of multiple athletes, and the processor is configured to derive, by using the run log, a prediction model that indicates a relationship between the measured value and the run time, and to predict the run time by using the prediction model and the expected value.

According to this aspect, the run time can be predicted with a simple method.

Preferably, the processor is configured to acquire the prediction model by performing multiple regression analysis using the measured value of each of the at least one item and the run time corresponding thereto for each athlete.

According to this aspect, the prediction model for predicting the run time can be acquired.

To achieve the above object, another aspect of the present invention provides a prediction method for predicting a run time of an athlete who competes in a race by driving a wheelchair (1), the prediction method being executed by a prediction device (21) comprising a storage device (24) and a processor (22), wherein the prediction method comprises: storing, in the storage device, a measured value of at least one item regarding a physical ability of an athlete who competes in the race and the run time of the athlete that were acquired in the past such that the measured value of the at least one item and the run time are associated with each other; acquiring, with the processor, an expected value corresponding to the at least one item; calculating, with the in processor, a predicted value of the run time corresponding to the expected value; and outputting the predicted value from the processor.

According to this aspect, it is possible to refer to the measured value of the at least one item regarding the physical ability of the athlete and the run time of the athlete that were acquired in the past, and therefore, the run time of the athlete can be properly predicted based on the expected value of the item(s) characterizing the physical ability of the athlete.

To achieve the above object, another aspect of the present invention provides a non-transitory storage medium, comprising a stored program, wherein the program, when executed by the processor, executes the aforementioned method.

According to this aspect also, the effects described above with regard to the method according to aspects of the present invention can be provided.

According to the foregoing arrangement, it is possible to provide a prediction device, a prediction method, and a prediction program which can properly predict a run time of an athlete who competes in a race by driving a wheelchair based on an expected value of an item characterizing the physical ability of the athlete.

### BRIEF DESCRIPTION OF THE DRAWINGS

FIG. 1 is a side view of a wheelchair and a measurement device;

FIG. 2 is a top view of the wheelchair and the measurement device;

FIG. 3 is a system configuration diagram of a prediction device;

FIG. 4 is a functional block diagram of the prediction device;

FIG. 5 shows an example of a run log;

FIGS. 6A to 6D are explanatory diagrams for explaining the movement of the athlete's arm when driving the wheelchair and a COP angle range;

FIG. 7 is a graph for explaining a method for gaining endurance ability;

FIG. 8 is a table showing combinations of explanatory variables and match rates acquired for the respective combinations; and

FIG. 9 is a schematic diagram showing an example of a display screen including change amount receiving fields.

### DETAILED DESCRIPTION OF THE INVENTION

In the following, an embodiment of a prediction device, a prediction method, and a prediction program according to the present invention will be described with reference to the drawings.

The prediction device, the prediction method, and the prediction program according to the present invention are used for the training of an athlete who competes in a race by driving a wheelchair 1, and are used to predict, before training, the record of the athlete after the training. The races for which record prediction may be performed by the prediction device, the prediction method, and the prediction program may include races in which athletes compete for the fastest time over a predetermined distance, such as track races, marathons, etc.

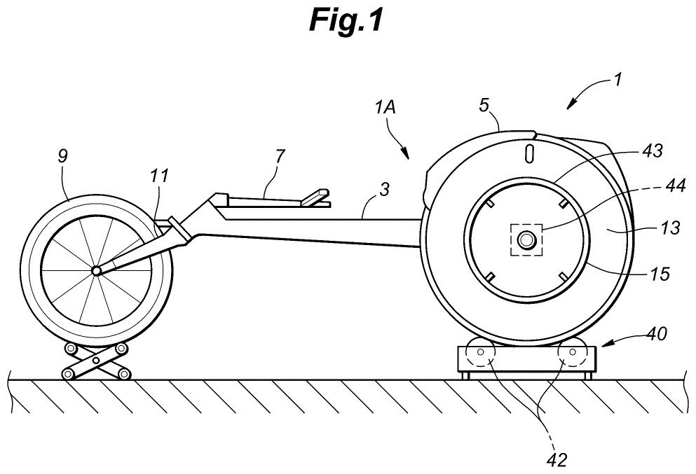

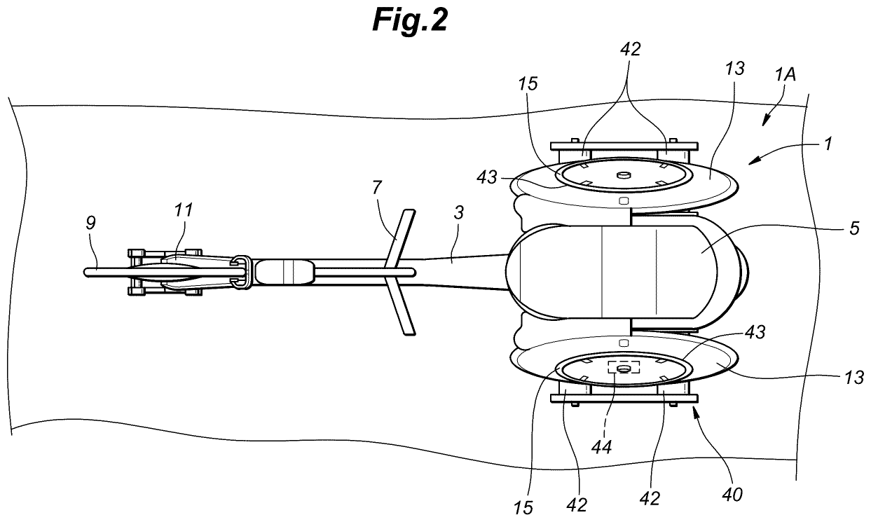

FIG. 1 is a side view of the wheelchair 1 for racing, and FIG. 2 is a top view of the wheelchair 1.

As shown in FIGS. 1 and 2, the wheelchair 1 includes a vehicle body frame 3, a cockpit 5 in which an occupant is to be seated, and a steering handle 7. A front portion of the vehicle body frame 3 is provided with a front wheel 9 (steered wheel) and a front fork 11. The rear portion of the vehicle body frame 3 is provided with a pair of left and right rear wheels 13 (driving wheels) and hand rims 15.

The rear wheels 13 are rotatably supported by the vehicle body frame 3 on left and right sides of the cockpit 5, respectively. The hand rims 15 are provided on sides of the respective rear wheels 13 opposite from the cockpit 5. When the athlete is seated in the cockpit 5 and pushes the hand rims 15 to apply a driving force, the rear wheels 13 rotate and the wheelchair 1 travels.

To execute the prediction method for predicting the record after training, a prediction device 21 executes the prediction program. Specifically, the prediction device 21 acquires an expected value (a value expected after training) of each of multiple items for indicating a physical ability of an athlete to be a target person, and based on the expected values, acquires and outputs a predicted value of the run time after the training. Thereby, the athlete or the coach who directs training of the athlete can figure out how the predicted value of the run time will change with the change of the expected values, and thus, can know the contents of training to be conducted. In the following, the athlete whose run time is to be predicted is referred to as a prediction target person.

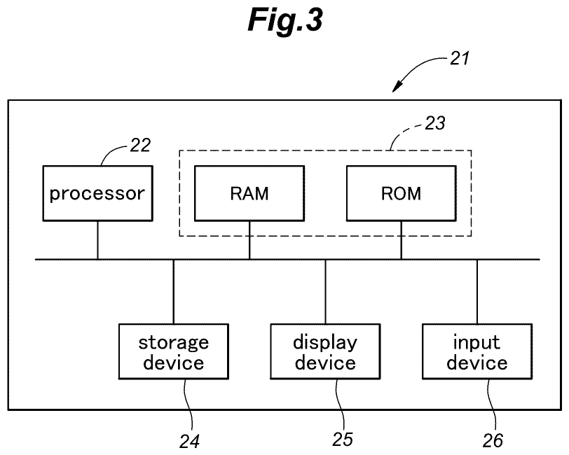

As shown in FIG. 3, the prediction device 21 is configured by a computer including a processor 22 such as a CPU or an MPU, a storage device 24 (may be also referred to as a storage) including a memory 23 (such as a RAM and a ROM), an SSD, a HDD, etc., a display device 25 such as a liquid crystal display, and an input device 26 such as a keyboard and a mouse.

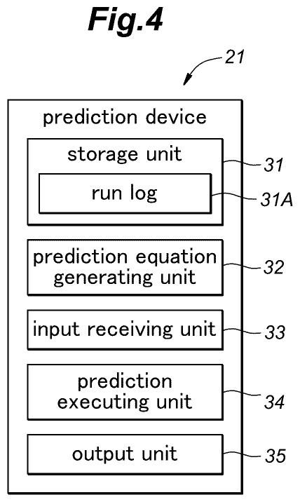

As shown in FIG. 4, the prediction device 21 includes a storage unit 31, a prediction equation generating unit 32, an input receiving unit 33, a prediction executing unit 34, and an output unit 35 as functional units for acquiring and outputting the predicted value.

The storage unit 31 may be configured by the storage device 24. The storage unit 31 stores a run log 31A. In the run log 31A, measured values of multiple items for indicating the physical ability of each athlete and the run time of each athlete that were acquired for each of multiple athletes in the past are recorded such that the run times are associated with the measured values of the multiple times.

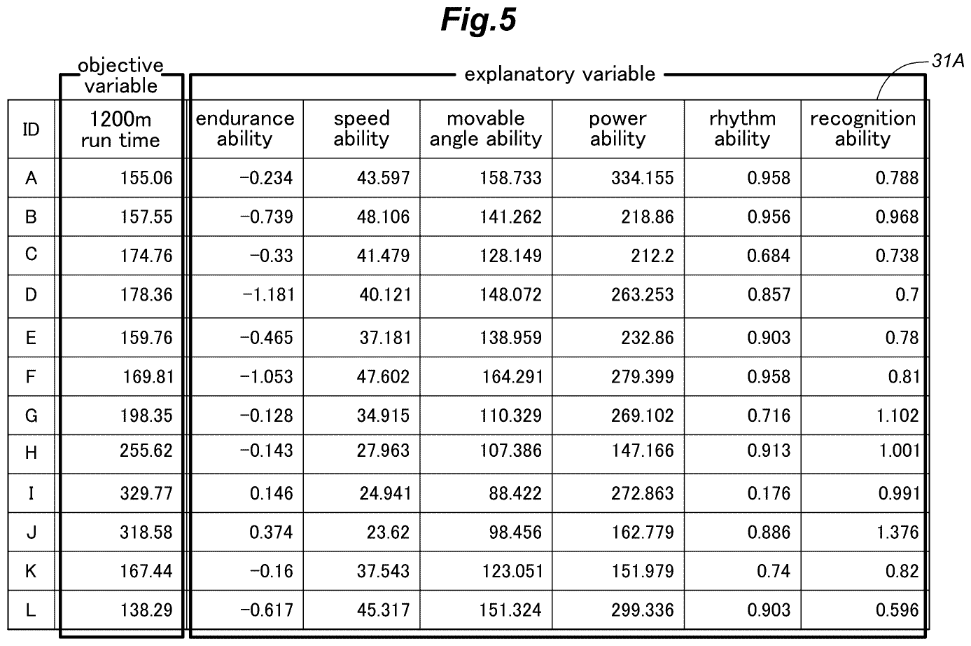

FIG. 5 shows an example of the run log 31A. The items for indicating the physical abilities of the athletes include at least five of six abilities consisting of a speed ability, a movable angle ability, an endurance ability, a rhythm ability, a recognition ability, and a power ability.

Note that the prediction device 21 may include a measurement wheelchair 1A for acquiring the measured values to be included in the run log 31A. In this case, the measured values corresponding to at least one of the items included in the run log 31A are acquired when the evaluator makes the athlete run in the measurement wheelchair 1A.

As shown in FIGS. 1 and 2, the measurement wheelchair 1A has substantially the same configuration as that of the wheelchair 1 which is configured for use in races, but further includes various sensors such as driving force sensors 43 for detecting driving forces applied to the hand rims 15, a rotation angle sensor 44 for detecting the rotation angle of the hand rims 15 (namely, the rotation angle of the rear wheels 13. Also called the rotation position), etc. Each driving force sensor 43 may include a strain gauge sensor provided on an outer surface of the corresponding hand rim 15. Preferably, the measurement wheelchair 1A is provided with a transmission device (not shown in the drawings) for outputting the detection results of the driving force sensors 43 and the rotation angle sensor 44 as wireless signals. Also preferably, the measurement wheelchair 1A is provided with a storage (not shown in the drawings) for holding/storing the detection results of the driving force sensors 43 and the rotation angle sensor 44.

The speed ability is an index indicating the speed of the athlete and is represented by the maximum rotation speed of the rear wheel 13 that the athlete can achieve.

FIG. 1 and FIG. 2 show, together with the wheelchair 1 (specifically, the measurement wheelchair 1A), an example of a measurement device 40 that is used when measuring the speed ability of the athlete.

The measurement device 40 includes an ergometer disposed to contact the respective rear wheels 13 of the wheelchair 1. The ergometer includes rollers 42 contacting the rear wheels 13 and motors (not shown in the drawings) for rotating the rollers 42, and torque meters (not shown in the drawings). The torque meters detect the rotation state (namely, torque, rotation speed, etc.) of the corresponding rollers 42.

In the present embodiment, the rotation speed of the roller 42 measured by the torque meter or the maximum value of the angular velocity (maximum speed) obtained from the detection result of the rotation angle sensor 44 included in the rear wheel 13 when the athlete is made to run at full speed in a state in which no load is generated from the motors is acquired as a feature quantity of the speed ability.

The movable angle ability is an index representing the hand movement range achieved by total body movement, and specifically corresponds to the region over which the athlete pushes (contacts) the hand rim 15 when rotating the rear wheel 13 one turn.

In the present embodiment, the movable angle ability is represented by a COP angle range. The COP (center of pressure) referred to here represents a point (applied position) that, when the athlete applies a driving force to the hand rim 15, becomes the center of the applied driving force (pressure).

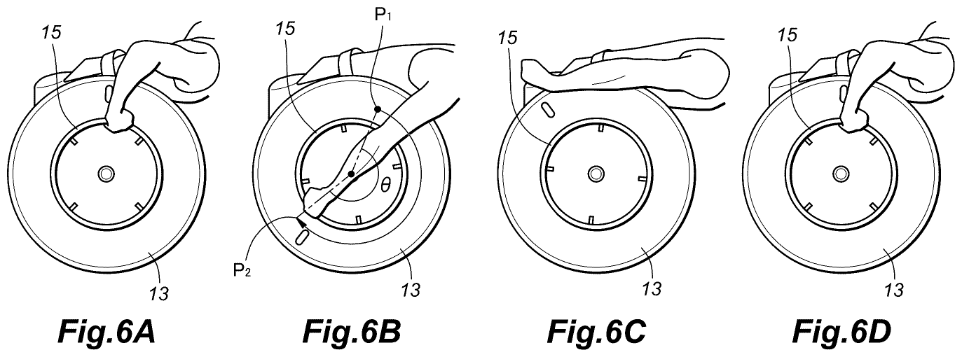

When driving the wheelchair 1, the athlete repeats the following process: touching the hand rim 15 (FIG. 6A), pushing the hand rim 15 to rotate (FIG. 6B), temporarily taking off the hand from the hand rim 15 (FIG. 6C), and pushing again the hand rim 15 (FIG. 6D). As shown in FIG. 6B, when the athlete runs the wheelchair 1 at full speed by rotating the rear wheel 13, the COP angle range θ is defined as an angle difference from a position P1 (contact start position) where the contact with the hand rim 15 is started to a position P2 (contact end position) where the contact with the hand rim 15 ends for each rotation of the rear wheel 13. Namely, the COP angle range θ is represented by an angle defined by a straight line connecting the axis center O of the rear wheel 13 to the position P1 and a straight line connecting the axis center O to the position P2. In this way, by using the COP angle range, the movable angle ability can be properly evaluated.

The COP angle range can be measured by using a driving force applied position estimation system configured to detect the COP. The driving force applied position estimation system may be configured by the driving force sensors 43 and the rotation angle sensor 44, for example. Also, the driving force applied position estimation system may be the one disclosed in JP7157723B2. For example, the COP angle range may be acquired by connecting the positions of the COP estimated by using the driving force applied position estimation system disclosed in JP7157723B2 when the athlete rotates the rear wheel 13 one turn. Further, the driving force applied position estimation system may be configured by using a computer (evaluation device) configured to calculate the COP angle range by estimating the rotation angle of the hand rim 15 based on the measurement result of the measurement device 40 and identifying the range over which the driving force was applied based on the detection result of the driving force sensor 43.

The endurance ability is an index representing the limit power that can last for a long time without fatigue, and corresponds to a feature quantity indicating the degree of decrease of the maximum speed depending on the travel distance.

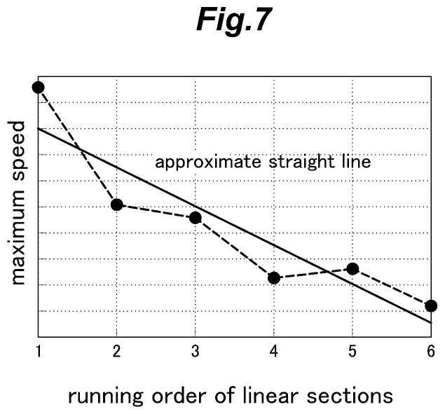

The endurance ability is acquired by making the athlete run around the track multiple times in the measurement wheelchair 1A and calculating the degree of increase in time it takes to run around the track or the degree of decrease of the maximum speed depending on the travel distance. In the present embodiment, the evaluator who evaluates the endurance ability makes the athlete run three laps of a 400 m track at full speed, and acquires the maximum speed in each of the six straight sections. Note that the maximum speed may be the one calculated from the rotation speed of the rear wheel 13 and the outer diameter of the rear wheel 13. Thereafter, as shown in FIG. 7, the evaluator plots the acquired maximum speeds on a graph with a horizontal axis representing the running order of the straight sections and a vertical axis representing the maximum speed, and acquires a tilt of a straight line obtained by linear fitting as the measured value of the endurance ability. In this way, by using the tilt indicating the degree of decrease, the endurance ability can be evaluated properly and conveniently.

The rhythm ability (also called the coordination ability) is an index representing the characteristics of the rhythm of operation when the athlete operates the rear wheels 13, and becomes greater as the change in the operation rhythm becomes smaller.

For example, the rhythm ability is represented by a correlation coefficient of waveforms indicating a time change of the driving force exerted by the athlete when rotating the rear wheel 13 of the wheelchair 1 one turn or a component of the driving force in the tangential direction of the hand rim 15. In the present embodiment, the evaluator who evaluates the rhythm ability makes the athlete run 300 m along the 400 m track in the wheelchair 1 provided with the driving force sensors 43 (the measurement wheelchair 1A). Then, by using the detection results of the driving force sensors 43, the evaluator acquires waveforms λ1 to λ4 (suffixes 1 to 4 indicate the running order) indicating the time change of the tangential force (the component of the driving force in the tangential direction of the hand rim 15) for four rotations (four strokes) of the rear wheel 13 before reaching the maximum speed in the straight section immediately before the end of the 300 m run.

Next, the evaluator acquires the correlation coefficient of each of the combinations (pairs) of waveforms adjoining in the running order, namely, the correlation coefficient of the waveforms 21 and 22, the correlation coefficient of the waveforms 22 and 23, and the correlation coefficient of the waveforms 23 and 24, and then acquires the average value of these correlation coefficients as a measured value indicating the rhythm ability. The measured value indicating the rhythm ability becomes greater as the waveforms become more similar (namely, the more constant the rhythm is). Note that the tangential force may be acquired by a six-axis force sensor (not shown in the drawings) similar to that shown in JP7157723B2 and provided between the hand rim 15 and the driving wheel (rear wheel 13). In this way, by using the correlation coefficients of the waveforms λ1 to λ4, the rhythm ability can be evaluated properly and conveniently.

The recognition ability is an index indicating the ability of the athlete to grasp the state of his/her own body and the surrounding situation and to perform operations precisely, and the recognition ability is related, for example, to a degree indicating how much the driving force exerted by the athlete contributes to the travel of the wheelchair 1.

In the present embodiment, the evaluator makes the athlete run 120 0m (three laps of a 400 m track) at full speed in the measurement wheelchair 1A, and acquires, by using the detection result of each driving force sensor 43, a tangential component of the driving force, namely, a tangential force, and a resultant force of the components of the driving force excluding the tangential component (hereinafter referred to as a hand rim pressing force or simply a pressing force). The evaluator calculates the recognition ability as a ratio of the tangential force to the hand rim pressing force, namely, according to the following equation: recognition ability=tangential force/hand rim pressing force. Thereby, the recognition ability can be evaluated properly and conveniently.

The power ability is an index indicating an instantaneous power and corresponds to the maximum tangential force that the athlete can exert (the maximum value of the component of the driving force in the tangential direction of the hand rim 15).

By making the athlete run 1200 m, 100 m, or 300 m at full speed in the wheelchair 1 provided with the driving force sensors 43 (the measurement wheelchair 1A), the evaluator acquires the tangential force for each stroke by using the detection result of each driving force sensor 43 and acquires the maximum value thereof as a feature quantity of the power ability.

The run times of the athletes included in the run log 31A may be run times in the race in which the prediction target person competes, such as 3 minute run distance and 1200-m sprint time. In the present embodiment, the race in which the prediction target person competes is 1200-m sprint, and the run times for 1200-m sprint are recorded for each athlete in the run log 31A.

The run log 31A includes the measured values of the endurance ability, the speed ability, the movable angle ability, the power ability, the rhythm ability, and the recognition ability of the prediction target person whose run time is to be predicted and the run time when the measurement was performed.

The prediction equation generating unit 32 may be configured by the memory 23, the storage device 24, and the processor 22. By using the run log 31A, the prediction equation generating unit 32 performs multiple regression analysis with the run time y being an objective variable (also called a response variable) and five or more of the measured values xᵢ (i=1 to 6) of the six items (abilities) for indicating the physical ability of the athlete being explanatory variables, thereby to calculate a regression equation. The regression equation is given by the equation (1) below.

$$y = {\sum }_{i = 1}^{6} {w}_{i} {x}_{i} + Z \qquad ( 1 )$$

In the equation (1), x₁, x₂, x₃, x₄, x₅, and x₆ respectively represent the measured value of the endurance ability, the measured value of the speed ability, the measured value of the movable angle ability, the measured value of the power ability, the measured value of the rhythm ability, and the measured value of the recognition ability, wᵢ (i=1 to 6) is a coefficient for each of the six abilities, and Z represents an intercept term.

To select an appropriate regression equation, the prediction equation generating unit 32 performs an evaluation process for each of all combinations each including five or more explanatory variables, and selects an optimal regression equation.

In the evaluation process, the prediction equation generating unit 32 first sets the coefficient wᵢ for the item (ability) other than the items selected as the explanatory variables to zero, and performs multiple regression analysis to acquire a regression equation (prediction model). When acquiring the regression equation, the prediction equation generating unit 32 divides the run log 31A into learning data and test data and acquires the regression equation by using only the learning data. The prediction equation generating unit 32 performs verification of consistency with the test data (cross-validation) by using the regression equation.

In the present embodiment, the run log 31A includes data for N athletes (N is an integer greater than or equal to 4), and the prediction equation generating unit 32 uses the data for two athletes as the test data and the data for the other N−2 athletes as the learning data. Subsequently, the prediction equation generating unit 32 generates the regression equation from the learning data, and by using the generated regression equation, acquires predicted values of run times of the two athletes corresponding to the test data, and determines whether the magnitude relationship between the predicted values matches the magnitude relationship between the measured values of run times of the two athletes corresponding to the test data.

The prediction equation generating unit 32 performs the selection of the test data for the all combinations, and performs the verification of consistency (namely, the determination of whether the magnitude relationship between the predicted values matches the magnitude relationship between the measured values) for each of them. The prediction equation generating unit 32 acquires the percentage of consistency (match rate) as an evaluation value of the regression equation (learning model).

The prediction equation generating unit 32 acquires the match rate for each of the all combinations each including five or more explanatory variables, and selects the regression equation with the highest match rate as the prediction equation.

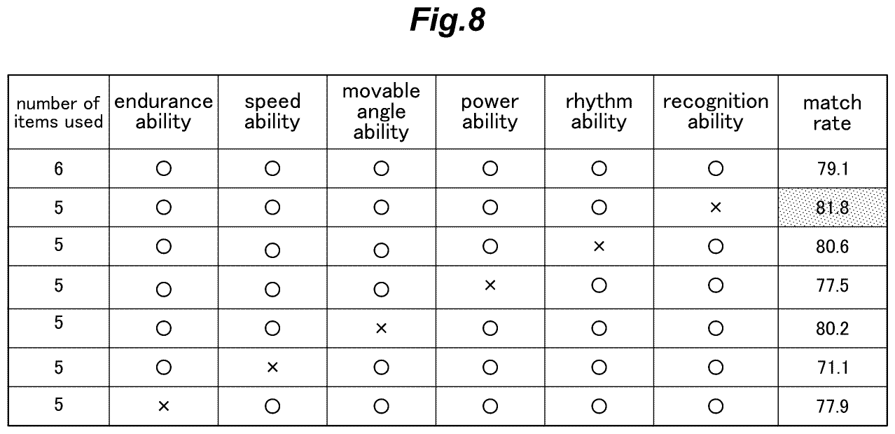

FIG. 8 shows various combinations of the explanatory variables and the match rate acquired for each combination. In the example of FIG. 8, the match rate in the case where the explanatory variables consist of five items excluding the rhythm ability is the highest (see the hatched part in FIG. 8). Thus, the prediction equation generating unit 32 acquires, as the prediction equation, the regression equation acquired in the case where the explanatory variables consist of five items excluding the rhythm ability.

In the present embodiment, the prediction equation generating unit 32 is configured to perform multiple regression analysis to acquire the prediction equation for acquiring a predicted value, but it is to be noted that the prediction equation is one example of prediction models that can be generated from the run log 31A to acquire a predicted value, and the prediction equation generating unit 32 may acquire the predicted value by using other various prediction models.

The input receiving unit 33 includes the memory 23, the processor 22, the display device 25, and the input device 26, and may be configured by executing a predetermined program on the processor 22. The input receiving unit 33 causes the display device 25 (display) to display a display screen 52 including receiving fields 50 for change amounts (also referred to as expected improvement amounts) of the items acquired as the explanatory variables.

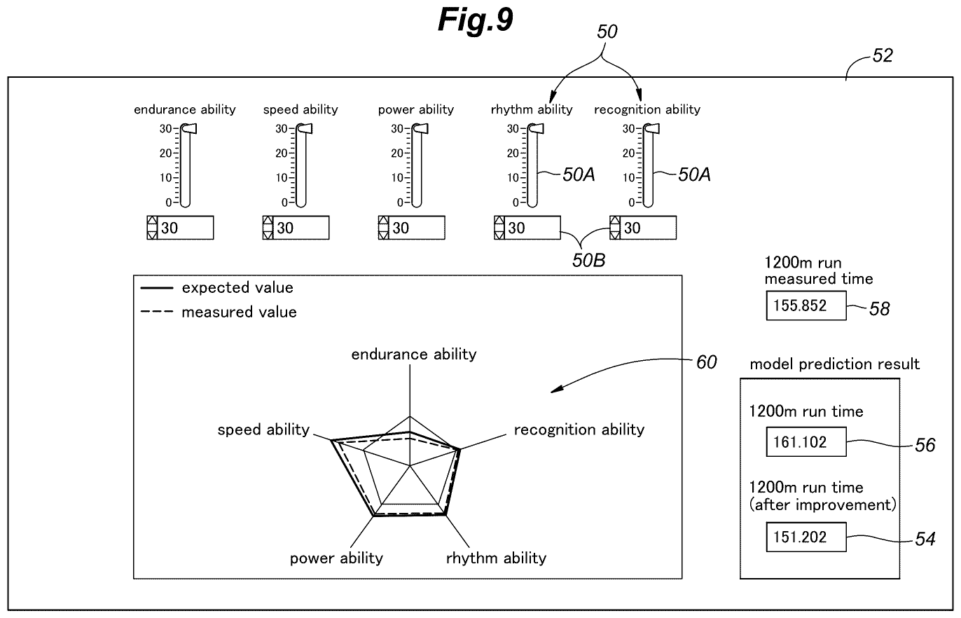

FIG. 9 shows an example of the display screen 52 including the receiving fields 50 for change amounts. The example shown in FIG. 9 corresponds to a case in which five items, the endurance ability, the speed ability, the power ability, the rhythm ability, and the recognition ability, are selected as the explanatory variables. The display screen 52 is provided with the receiving fields 50 for change amounts for the five items selected as the explanatory variables. Each of the receiving fields 50 is configured by a slider 50A for receiving an input of an increase rate (%) and an input field 50B for allowing input of a numeric value of the increase rate. When the display screen 52 is displayed, the value of each slider 50A is set to 0 (the increase rate is 0).

Upon input of the change amounts, the input receiving unit 33 acquires, from the run log 31A, the measured values of the items corresponding to the explanatory variables for the prediction target person, and calculates the expected values in which the change amounts inputted to the receiving fields 50 are reflected. In the present embodiment, since the input receiving unit 33 receives increase rates through the respective receiving fields 50, the input receiving unit 33 acquires the expected values by multiplying the measured values of the items corresponding to the explanatory variables acquired for the prediction target person by the values obtained by adding 1 to the corresponding increase rates divided by 100. Upon completion of the acquisition of the expected values, the input receiving unit 33 outputs the measured values of the items corresponding to the explanatory variables and the corresponding expected values to the prediction executing unit 34.

The prediction executing unit 34 may be configured by executing a predetermined program on the processor 22. Upon acquisition of the expected values for the respective items corresponding to the explanatory variables from the input receiving unit 33, the prediction executing unit 34 puts the expected values into the regression equation represented by the equation (1), and thereby acquires the predicted value of the run time.

The prediction executing unit 34 outputs the predicted value of the run time that is acquired to the output unit 35. In the present embodiment, the prediction executing unit 34 outputs, together with the predicted value of the run time, the measured values of the items corresponding to the explanatory variables and the corresponding expected values to the output unit 35.

The output unit 35 may be configured by the display device 25. Upon acquisition of the predicted value of the run time from the prediction executing unit 34, the output unit 35 displays the predicted value of the run time in a display field 54 provided in the display screen 52.

In the present embodiment, the display screen 52 is provided with a display field 56 for displaying the predicted value of the run time calculated by using the regression equation based on the measured values of the items corresponding to the explanatory variables, and a display field 58 for displaying the measured value of the run time at that time.

In the present embodiment, the output unit 35 further displays the measured value of each item acquired from the prediction executing unit 34 and the expected value of the same on the display screen 52. In the example shown in FIG. 9, since the five items consisting of the endurance ability, the speed ability, the power ability, the rhythm ability, and the recognition ability are selected as the explanatory variables, the measured values (broken line) and the expected values (thick line) of them are shown by a radar chart 60. In addition, ideal values (solid line) of the five explanatory variables are shown in the radar chart 60.

Next, the operation and the effects of the prediction device 21, the prediction method, and the prediction program configured as above will be described.

In the storage device 24 of the prediction device 21, the measured values of the multiple items related to the physical ability of each athlete and the run time of each athlete that were acquired in the past are stored as the run log 31A such that the run time is associated with the measured values of the multiple items.

When the display screen 52 is displayed, the processor 22 executes the prediction program to perform the prediction method. The processor 22 (the prediction executing unit 34) first acquires change amounts from the input values to the receiving fields 50 (in this embodiment, the input values are increase rates, whose initial values are 0), and calculates the expected value of each of the items corresponding to the explanatory variables. Thereafter, the processor 22 (the prediction executing unit 34) calculates the predicted value of the run time of the prediction target person, and displays the predicted value in the display field 54 of the display (the output unit 35).

The items corresponding to the explanatory variables include at least five of the six abilities consisting of the speed ability, the movable angle ability, the endurance ability, the rhythm ability, the recognition ability, and the power ability. As shown in FIG. 8, it can be understood that when five or more of these items are included as the explanatory variables, the match rate exceeds 70%. Therefore, by using, as the items corresponding to the explanatory variables, five or more of the speed ability, the movable angle ability, the endurance ability, the rhythm ability, the recognition ability, and the power ability, it is possible to predict the run time.

When the athlete or the coach operates the sliders 50A provided in the display screen 52 (or inputs numerical values in the input fields 50B), the processor 22 (the input receiving unit 33, the prediction executing unit 34) acquires the change amounts and calculates the expected values, and causes the display (the output unit 35) to display the measured values and the expected values on the radar chart 60. Further, the processor 22 (the prediction executing unit 34) puts the expected values in the regression equation represented by the equation (1) to calculate the predicted value of the run time, and displays the predicted value of the run time on the display screen 52 of the display (the output unit 35).

When the athlete or the coach selects an item and operates the slider 50A (or inputs a numerical value in the input field 50B), the run time changes. Therefore, the athlete or the coach can understand how the run time depends on the selected item. Thus, the athlete and/or the coach can understand the contents of training that the athlete should do.

Concrete embodiments have been described in the foregoing, but the present invention can be modified in various ways without being limited to the above embodiments.

The present invention is not limited by the number of data included in the run log 31A (namely, the value of N) or the number of test data and the learning data. The consistency verification method in the above embodiment is only one example, and various known methods may be adopted.

In the above embodiment, the measured value of the rhythm ability was calculated as the average value of the correlation coefficient of the waveforms λ1 and λ2, the correlation coefficient of the waveforms λ2 and λ3, and the correlation coefficient of the waveforms λ3 and λ4, but it is also possible to calculate the correlation coefficients of the all possible pairs of the waveforms λ1 to λ4 and to calculate the measured value of the rhythm ability as the average value thereof.

In the above embodiment, the measured value of the recognition ability was calculated as a ratio of the tangential component of the driving force to the hand rim pressing force, but the measured value of the recognition ability may be calculated as a ratio of the tangential component of the driving force to the magnitude of the driving force.

In the above embodiment, the items corresponding to the explanatory variables included at least five of the six consisting of the speed ability, the movable angle ability, the endurance ability, the rhythm ability, the recognition ability, and the power ability, but the present invention is not limited to this embodiment. For example, the items corresponding to the explanatory variables need to include one of the six abilities consisting of the speed ability, the movable angle ability, the endurance ability, the rhythm ability, the recognition ability, and the power ability, and preferably include at least four of them.

Also, in the above embodiment, the prediction device 21 was configured to output the predicted value of the run time for 1200-m sprint, but the target to be predicted by the prediction device 21 is not limited to this. The prediction device 21 may be configured to predict the run time for 300-m sprint or 500-m sprint, the travel distance for 3 minute run, or the like.

1. A prediction device for predicting a run time of a target person who competes in a race by driving a wheelchair, the prediction device comprising:
a storage device configured to store a measured value of at least one item regarding a physical ability of an athlete who competes in the race and the run time of the athlete that were acquired in the past such that the measured value of the at least one item and the run time are associated with each other; and
a processor configured to acquire an expected value corresponding to the at least one item, to calculate a predicted value of the run time corresponding to the expected value, and to output the predicted value.

2. The prediction device according to claim 1, wherein the at least one item includes at least one of six abilities consisting of a speed ability, a movable angle ability, an endurance ability, a rhythm ability, a recognition ability, and a power ability.

3. The prediction device according to claim 2, wherein the movable angle ability is acquired from an angle difference from a contact start position where the athlete starts touching a hand rim of the wheelchair to a contact end position where contact with the hand rim ends.

4. The prediction device according to claim 2, wherein the endurance ability is acquired by making the athlete run around a track multiple times in the wheelchair and calculating a degree of increase in time it takes to run around the track.

5. The prediction device according to claim 2, wherein the rhythm ability is acquired by calculating a correlation coefficient of waveforms each showing a time change of a driving force applied by the athlete when rotating a rear wheel of the wheelchair one turn or a component of the driving force in a tangential direction of a hand rim of the wheelchair.

6. The prediction device according to claim 2, wherein the recognition ability is acquired from a ratio between a tangential force which is a component of a driving force exerted by the athlete in a tangential direction of a hand rim of the wheelchair and a pressing force which is a resultant force of components of the driving force excluding the component in the tangential direction of the hand rim.

7. The prediction device according to claim 2, wherein the at least one item includes at least four of the six abilities consisting of the speed ability, the movable angle ability, the endurance ability, the rhythm ability, the recognition ability, and the power ability.

8. The prediction device according to claim 1, wherein the prediction device comprises the wheelchair provided with a vehicle body frame, a rear wheel, a hand rim provided on the rear wheel, and a sensor configured to detect a force applied to the hand rim and a rotation position of the hand rim, and
the prediction device acquires the measured value by making the athlete run using the wheelchair.

9. The prediction device according to claim 1, wherein the storage device is configured to store a run log in which the measured value of each of the at least one item and the run time corresponding thereto are recorded so as to be associated with each other for each of multiple athletes, and
the processor is configured to derive, by using the run log, a prediction model that indicates a relationship between the measured value and the run time, and to predict the run time by using the prediction model and the expected value.

10. The prediction device according to claim 9, wherein the processor is configured to acquire the prediction model by performing multiple regression analysis using the measured value of each of the at least one item and the run time corresponding thereto for each athlete.

11. A prediction method for predicting a run time of a target person who competes in a race by driving a wheelchair, the prediction method being executed by a prediction device comprising a storage device and a processor, wherein the prediction method comprises:
storing, in the storage device, a measured value of at least one item regarding a physical ability of an athlete who competes in the race and the run time of the athlete that were acquired in the past such that the measured value of the at least one item and the run time are associated with each other;
acquiring, with the processor, an expected value corresponding to the at least one item;
calculating, with the processor, a predicted value of the run time corresponding to the expected value; and
outputting the predicted value from the processor.

12. A non-transitory computer-readable storage medium, comprising a stored program, wherein the program, when executed by the processor, executes the method of claim 11.
